SEQUENCE LISTING (10 sequences)

SEQ ID NO: 1 - DNA, 19 bp, synthetic construct
  source 1..19: mol_type = other DNA; note = Forward primer of the primer GAPDH
gaaggtgaaggtcggagtc

SEQ ID NO: 2 - DNA, 20 bp, synthetic construct
  source 1..20: mol_type = other DNA; note = Reverse primer of the primer GAPDH
gaagatggtgatgggatttc

SEQ ID NO: 3 - DNA, 23 bp, synthetic construct
  source 1..23: mol_type = other DNA; note = Forward primer of the primer NOX2
gcaatggtgtgaatcgcagagtg

SEQ ID NO: 4 - DNA, 25 bp, synthetic construct
  source 1..25: mol_type = other DNA; note = Reverse primer of the primer NOX2
tcgaagacaactggacaggaatctc

SEQ ID NO: 5 - DNA, 22 bp, synthetic construct
  source 1..22: mol_type = other DNA; note = Forward primer of the primer TNF-alpha
tggcgtggagctgagagataac

SEQ ID NO: 6 - DNA, 21 bp, synthetic construct
  source 1..21: mol_type = other DNA; note = Reverse primer of the primer TNF-alpha
gctgatggtgtgggtgaggag

SEQ ID NO: 7 - DNA, 21 bp, synthetic construct
  source 1..21: mol_type = other DNA; note = Forward primer of the primer IL-6
ttcggtccagttgccttctcc

SEQ ID NO: 8 - DNA, 22 bp, synthetic construct
  source 1..22: mol_type = other DNA; note = Reverse primer of the primer IL-6
tctgaagaggtgagtggctgtc

SEQ ID NO: 9 - DNA, 25 bp, synthetic construct
  source 1..25: mol_type = other DNA; note = Forward primer of the primer IL-1beta
atggcttattacagtggcaatgagg

SEQ ID NO: 10 - DNA, 22 bp, synthetic construct
  source 1..22: mol_type = other DNA; note = Reverse primer of the primer IL-1beta
agtggtggtcggagattcgtag

(19) United States
(12) Patent Application Publication
(10) Pub. No.: US 2025/0360096 A1
(43) Pub. Date: Nov. 27, 2025

(54) USE OF COMPOUND K67 IN PREPARATION OF ANTI-TUBERCULOSIS DRUG

(71) Applicant: Bengbu Medical University, Bengbu City (CN)

(72) Inventors: Jingzhu LYU, Bengbu City (CN); Zhongqing QIAN, Bengbu City (CN); Geman XIA, Bengbu City (CN); Xiaoyu ZHOU, Bengbu City (CN); Ru HUANG, Bengbu City (CN); Huiting DAI, Bengbu City (CN); Tong FENG, Bengbu City (CN); Danrui LIU, Bengbu City (CN)

(73) Assignee: Bengbu Medical University, Bengbu City (CN)

(21) Appl. No.: 19/173,524

(22) Filed: Apr. 8, 2025

(30) Foreign Application Priority Data
May 23, 2024 (CN) 202410659027.5

(51) Int. Cl. A61K 31/18; A61P 31/06

(52) U.S. Cl. CPC A61K 31/18; A61P 31/06

(57) ABSTRACT

Use of a compound K67 in preparation of an anti-tuberculosis drug is provided, belonging to the technical field of chemical drugs. Experiments show that the compound K67 can inhibit proliferation of Mycobacterium tuberculosis, promote macrophage autophagy, and enhance a bactericidal effect. The compound can also reduce an oxidative stress level of the macrophages, inhibit expression of genes and proteins of inflammatory factors in the macrophages, and alleviate an inflammatory damage caused by infection of the Mycobacterium tuberculosis. Therefore, the compound K67 shows a potential research and application value in the field of anti-tuberculosis.

### CROSS-REFERENCE TO RELATED APPLICATION

This patent application claims the benefit and priority of Chinese Patent Application No. 202410659027.5 filed with the China National Intellectual Property Administration on May 23, 2024, the disclosure of which is incorporated by reference herein in its entirety as part of the present application.

### REFERENCE TO SEQUENCE LISTING

A computer-readable XML file entitled “GWP20241208064_Sequence listing”, which was created on Feb. 10, 2025, with a file size of about 11,714 bytes, contains the sequence listing for this application, has been filed with this application, and is hereby incorporated by reference in its entirety.

### TECHNICAL FIELD

The present disclosure belongs to the technical field of chemical drugs, and specifically relates to the use of compound K67 in preparation of an anti-tuberculosis drug.

### BACKGROUND

In recent years, anti-tuberculosis drugs have been widely used and abused. The combined use of some first-line anti-tuberculosis drugs, such as isoniazid (INH), ethambutol (EMB), rifampicin (RIF), and pyrazinamide (PZA), can exert a better anti-tuberculosis effect, but the abuse of drugs makes pathogens prone to developing drug resistance. In terms of second-line anti-tuberculosis drugs, only two novel drugs, bedaquiline and delamanid, have been launched in recent decades^{[1]}, and are prone to causing adverse drug reactions. The emergence of drug-resistant strains and the many side effects of drug use have made the task of developing novel anti-tuberculosis drugs urgent^{[2]}.

Macrophages, as sentinel cells that detect invading microorganisms, have strong phagocytic ability and are also the main target cells for Mycobacterium tuberculosis (M. tb) infection. When M. tb infects macrophages, the macrophages can phagocytize M. tb to produce an immune response, while generating a large amount of reactive oxygen species (ROS) and then synthesizing and secreting a variety of pro-inflammatory cytokines, such as tumor necrosis factor (TNF)-α, interleukin (IL)-6, and IL-1β. At this time, macrophages kill intracellular M. tb through autophagy, strong self-oxidation, and other pathways. However, a high ROS level in macrophages can induce oxidative stress damage, and the secretion of pro-inflammatory cytokines can also activate a variety of immune cells to promote the occurrence and development of inflammatory responses.

The compound, 2-acetyl-1,4-di[(4-methoxyphenylsulfonyl)amino]naphthalene (K67), is a small-molecule compound obtained by Japanese scholars through high-throughput screening and identification^{[3]}. Limited experimental data show that this compound can not only inhibit the proliferation of liver cancer cells and anticancer drug tolerance^{[4]}, but also inhibit the proliferation of esophageal squamous cell carcinoma cells and enhance radiotherapy sensitivity ^{[5]}. However, this compound has not yet been used in the field of anti-tuberculosis.

### Cited References

[1] Wu Jiang, Cui Jiawei, Xue Yunxin, Wang Dai: Research progress on anti-tuberculosis drug targets. Chinese Journal of Antibiotics, 2022, 47 (11): 1119-1127.

[2]. Liang Chen, Tang Shenjie, Lin Minggui: Research progress on comprehensive treatment of tuberculosis. Journal of Tuberculosis and Lung Disease: 1-23.

[3]. Yasuda D, Obata R, Takahashi K, Ohe T, Mashino T: [Hit-to-Lead in Academia: Discovery of a Protein-Protein Interaction Inhibitor of Keap1-Nrf2]. Yakugaku Zasshi, 2018, 138 (8): 1059-1065.

[4]. Saito T, Ichimura Y, Taguchi K, Suzuki T, Mizushima T, Takagi K, Hirose Y, Nagahashi M, Iso T, Fukutomi T et al: p62/Sqstm1 promotes malignancy of HCV-positive hepatocellular carcinoma through Nrf2-dependent metabolic reprogramming. Nat Commun, 2016, 7:12030.

[5]. Wang Zhe: Research on small-molecule compound K67 mediating radiosensitization of esophageal squamous cell carcinoma by regulating p62-Keap1-Nrf2 pathway. Doctoral dissertation. Shandong University; 2019.

### SUMMARY

A purpose of the present disclosure is to provide the use of a compound K67 in preparation of an anti-tuberculosis drug. The compound K67 can not only inhibit proliferation of Mycobacterium tuberculosis, but also increase an autophagy level of macrophages, reduce an oxidative stress level of the macrophages, and inhibit secretion of pro-inflammatory factors by the macrophages, thereby reducing the degree of inflammatory damage to a body.

The present disclosure provides the use of a compound K67 in preparation of an anti-tuberculosis drug.

Preferably, the anti-tuberculosis drug inhibits proliferation of Mycobacterium tuberculosis in a body.

The present disclosure further provides the use of a compound K67 in preparation of a drug for repairing a body damage caused by mycobacterium infection.

Preferably, the compound K67 regulates at least one of the following reactions: increasing an autophagy level of macrophages, reducing an oxidative stress level of the macrophages, and inhibiting an inflammatory response in the macrophages; and the macrophages are Mycobacterium tuberculosis-infected macrophages.

Preferably, the increasing the autophagy level of the macrophages includes promoting expression of an autophagy-related protein and/or increasing an LC3 II/LC3 I ratio.

Preferably, the reducing the oxidative stress level of the macrophages includes reducing a level of ROS in the macrophages and/or reducing an expression level of nicotinamide adenine dinucleotide phosphate (NADPH) oxidase 2 (NOX2) in the macrophages.

Preferably, the inhibiting the inflammatory response in the macrophages includes inhibiting expression of a gene and/or a protein of a pro-inflammatory factor in the macrophages.

Preferably, the pro-inflammatory factor is at least one selected from the group consisting of TNF-α, IL-6, and IL-1β.

The present disclosure further provides use of a compound K67 in preparation of an antibacterial agent for inhibiting a mycobacterium in vitro.

Preferably, the mycobacterium is Mycobacterium tuberculosis.

### Beneficial Effects

The present disclosure provides use of a compound K67 in preparation of an anti-tuberculosis drug. In the present disclosure, the examples show that the compound K67 has the effect of inhibiting the proliferation of Mycobacterium tuberculosis (M. tb) in vitro. The compound K67 can effectively kill M. tb in macrophages at the cellular level, reduce the load of M. tb in macrophages, promote the expression of autophagy-related proteins p-p62 and p62, and increase the LC3 II/LC3 I ratio, thereby increasing the autophagy level of macrophages. This compound can also reduce the level of ROS and the expression level of NOX2 in macrophages, inhibit the expression of genes and proteins of pro-inflammatory factors such as TNF-α, IL-6, and IL-1B in macrophages, and reduce the inflammatory damage caused by M. tb infection. Moreover, it is verified that the compound K67 has no cytotoxicity to macrophages at a concentration of 0 μM to 20 μM and shows desirable safety in use. In addition, it is verified that the compound K67 does not affect the phagocytic function of THP-1-derived macrophages against M. tb. Accordingly, the compound K67 has the potential application value in preparing a novel anti-tuberculosis drug.

### BRIEF DESCRIPTION OF THE DRAWINGS

To describe the technical solutions in examples of the present disclosure or in the prior art more clearly, the accompanying drawings required in the examples are briefly described below. Apparently, the accompanying drawings in the following description show merely some examples of the present disclosure, and other drawings can still be derived from these accompanying drawings by those of ordinary skill in the art without creative efforts.

FIG. 1 shows a survival rate of macrophages after K67 treatment detected by CCK8 assay.

FIG. 2A-FIG. 2B show an extracellular K67 antibacterial experiment; where FIG. 2A shows the growth of M. tb colonies on the plate before and after the action of K67, and FIG. 2B shows the colony count (CFU×10⁴) and statistical analysis of FIG. 2A.

FIG. 3 shows an effect of K67 treatment on the phagocytic function of macrophages; where MFI represents mean fluorescence intensity, reflecting the phagocytic function of THP-1-derived macrophages on M. tb.

FIG. 4A-FIG. 4B show a killing activity of macrophages pretreated with K67 on intracellular M. tb; where FIG. 4A shows an effect of K67 pretreatment of macrophages on the growth of M. tb colonies on the plate, and FIG. 4B shows the colony count (CFU×10⁴) and statistical analysis of FIG. 4A.

FIG. 5A-FIG. 5B show a killing activity of K67 against M. tb in infected macrophages; where FIG. 5A shows an effect of K67 treatment on the growth of M. tb colonies on the plate in infected macrophages, and FIG. 5B shows the colony count (CFU×10⁴) and statistical analysis of FIG. 5A.

FIG. 6A-FIG. 6B show an effect of K67 pretreatment on autophagy in macrophages; where FIG. 6A shows an effect of K67 pretreatment on the expression level of autophagy-related proteins in macrophages by Western blot, and FIG. 6B shows the statistical analysis of the grayscale value of immunoblot in FIG. 6A; LC3 II represents a ratio of LC3 II/LC3 I.

FIG. 7 shows a statistical analysis on the MFI of ROS levels determined by flow cytometry.

FIG. 8 shows the statistical analysis of the mRNA expression levels of NOX2.

FIG. 9A-FIG. 9F show an effect of K67 pretreatment of macrophages on the expression levels of pro-inflammatory cytokines TNF-α, IL-6, and IL-1β; where FIG. 9A-FIG. 9C show the statistical analysis of the mRNA expression level of each pro-inflammatory cytokine detected by RT-qPCR, and FIG. 9D-FIG. 9F show the statistical analysis of the expression level of each pro-inflammatory cytokine in a cell culture supernatant detected by ELISA;

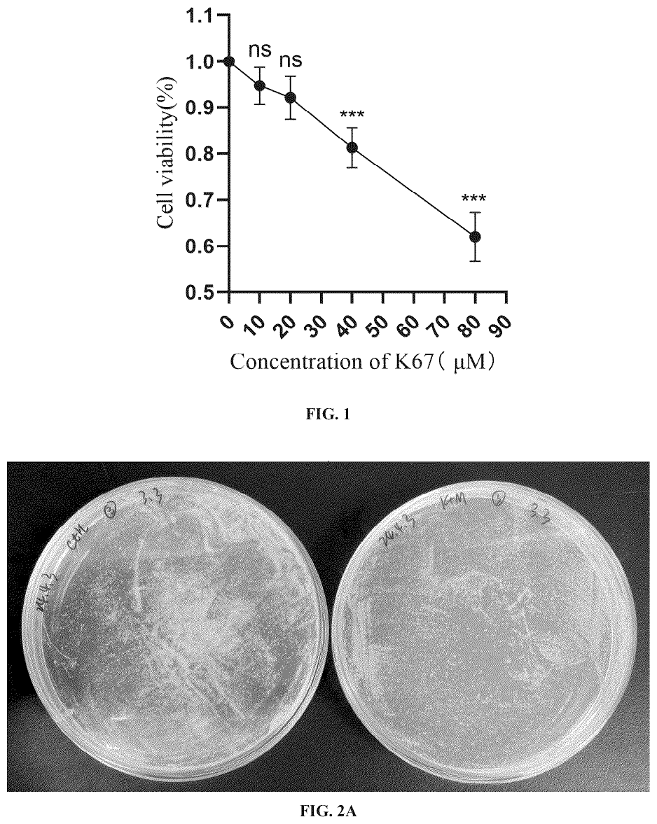

NOTE: in FIG. 1 through FIG. 9F, n=3, * indicates P<0.05, ** indicates P<0.01, * indicates P<0.001.

### DETAILED DESCRIPTION OF THE EMBODIMENTS

The present disclosure provides the use of a compound K67 in preparation of an anti-tuberculosis drug.

In the present disclosure, the anti-tuberculosis drug preferably inhibits proliferation of Mycobacterium tuberculosis in a body.

In the present disclosure, the compound K67 is 2-acetyl-1,4-di[(4-methoxyphenylsulfonyl)amino]naphthalene, has a molecular formula of C₂₉H₃₀N₂O₇S₂, a molecular weight of 582.69, and a structural formula shown in Formula I.

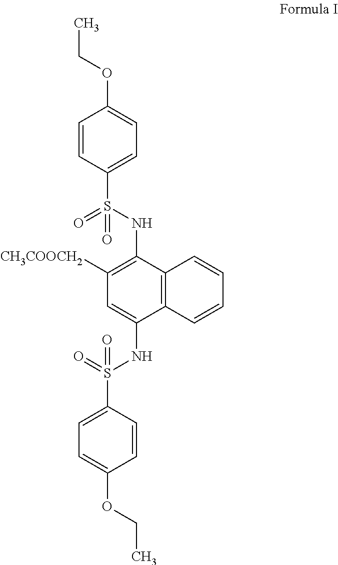

In the examples of the present disclosure, the compound K67 is purchased from Sigma-Aldrich with a product number of SML1922, and is a powdery substance that can be dissolved in DMSO.

In an example of the present disclosure, an effect of the compound K67 on the activity of THP-1-derived macrophages is detected. Different doses of the compound K67 are added into THP-1-derived macrophages to make their final concentrations 10 μM, 20 μM, 40 μM, and 80 μM, respectively. The results show that within the concentrations of 0 μM to 20 μM, the compound K67 has no cytotoxicity to macrophages and shows desirable safety for use. An effect of the compound K67 on the phagocytic function of THP-1-derived macrophages on Mycobacterium tuberculosis is further verified. After the compound K67 is added into THP-1-derived macrophages for culture, the THP-1-derived macrophages are infected with RFP-H37Ra bacterial suspension, and a content of RFP-H37Ra in macrophages is detected by flow cytometry. The results show that the compound K67 does not affect the phagocytic function of THP-1-derived macrophages on M. tb.

In an example of the present disclosure, an experiment is also conducted on a killing effect of the compound K67 on M. tb in macrophages. THP-1-derived macrophages are treated with 20 μM K67 and then infected with RFP-H37Ra bacterial suspension to detect the effect of K67 on M. tb in macrophages. The results show that the number of M. tb colonies on the agar plate of the group treated with K67 is significantly reduced, indicating that the K67 can act on macrophages that are not infected with M. tb, regulate the immune response of macrophages to M. tb, thereby effectively killing M. tb that has invaded macrophages in the later stage and reducing the load of M. tb in macrophages. Moreover, THP-1-derived macrophages are infected with RFP-H37Ra bacterial suspension and then treated with 20 μM K67 to detect the effect of K67 on M. tb in macrophages. The results show that the number of colonies on the agar plate of the group infected and then treated with K67 is significantly reduced, indicating that K67 can also act on macrophages that have been infected with M. tb, change the immune response of infected macrophages to M. tb, thereby effectively killing M. tb that has invaded macrophages and reducing the load of M. tb in macrophages.

In the present disclosure, the compound K67 regulates at least one of the following reactions: increasing an autophagy level of macrophages, reducing an oxidative stress level of the macrophages, and inhibiting an inflammatory response in the macrophages; and the macrophages are Mycobacterium tuberculosis-infected macrophages. The increasing the autophagy level of the macrophages preferably includes promoting expression of an autophagy-related protein and/or increasing an LC3 II/LC3 I ratio; the promoting the expression of autophagy-related proteins preferably includes promoting the expression of autophagy-related proteins p-p62 and p62. Autophagy is an important means of defense for macrophages, and macrophages can kill invading pathogenic microorganisms through the autophagy. During Mycobacterium tuberculosis infection, autophagy not only plays an important role in the antibacterial mechanism of intracellular pathogen clearance, but also prevents excessive inflammation to avoid causing damages to the host.

In an example of the present disclosure, an effect of the compound K67 is detected on autophagy of THP-1-derived macrophages after infection with M. tb. After pre-treatment of THP-1-derived macrophages with K67, H37Ra bacterial suspension is added to infect the THP-1-derived macrophages. The results of detecting the expression levels of autophagy-related proteins show that compared with the control group, there is no significant difference in the expression of autophagy-related proteins in the K67 group alone (P>0.05), while the expression of autophagy-related proteins in the K67+M. tb group is increased compared with the M. tb infection group alone, and the expression of autophagy-related proteins p-p62 and p62 and/or the ratio of LC3 II/LC3 I are significantly increased. This indicates that the compound K67 can enhance the autophagy level of macrophages after infection with M. tb and improve the cellular immune response against M. tb.

In the present disclosure, the reducing the oxidative stress level of the macrophages preferably includes reducing a level of ROS in the macrophages and/or reducing an expression level of NOX2 in the macrophages. In an example, an effect of K67 on the ROS level of THP-1-derived macrophages is detected. The ROS level of THP-1-derived macrophages pretreated with K67 is detected by flow cytometry, and the results show that K67 can significantly inhibit the production of ROS in macrophages regardless of whether they are infected with M. tb. This indicates that K67 can reduce the oxidative stress level caused by macrophages themselves and M. tb infection to cells, and play a protective role on cells. An effect of the compound K67 is further determined on the mRNA expression level of NADPH oxidase 2 (NOX2) in THP-1-derived macrophages infected with M. tb. The results show that compared with the control group, M. tb infection leads to a significant up-regulation of NOX2 mRNA expression (P<0.05), while the K67 significantly down-regulates the high expression of NOX2 mRNA caused by M. tb infection (P<0.05). Therefore, the compound K67 can reduce the expression level of NOX2 in macrophages infected with M. tb and inhibit the oxidative stress response caused by M. tb infection.

In the present disclosure, the inhibiting the inflammatory response in the macrophages preferably includes inhibiting expression of a gene and/or a protein of a pro-inflammatory factor in the macrophages. The pro-inflammatory factor is preferably at least one selected from the group consisting of TNF-α, IL-6, and IL-1β; the gene preferably includes mRNA. In an example, an effect of the compound K67 is determined on the mRNA and protein expression levels of TNF-α, IL-6, and IL-1βafter THP-1-derived macrophages are infected with M. tb. The results show that M. tb infection causes a significant increase in the mRNA and protein expression levels of TNF-α, IL-6, and IL-1β(P<0.001), while the K67 significantly down-regulates the high mRNA and protein expression levels of TNF-α, IL-6, and IL-1βcaused by M. tb infection. This suggests that the compound K67 can inhibit the high inflammatory state caused by M. tb infection and protect cells from damage by oxidative stress. Accordingly, the compound K67 has the potential application value in preparing a novel anti-tuberculosis drug.

Based on the fact that the compound K67 has the function of inhibiting the proliferation of Mycobacterium tuberculosis and also has the performance of reducing body damage, the present disclosure further provides the use of the compound K67 in preparation of a drug for repairing a body damage caused by mycobacterium infection.

The present disclosure further provides the use of the compound K67 in preparation of an antibacterial agent for inhibiting a mycobacterium in vitro.

In the present disclosure, the mycobacterium is preferably Mycobacterium tuberculosis.

In the present disclosure, a dosage form of the antibacterial agent is preferably selected from the group consisting of a powder and an aqueous solution, more preferably the aqueous solution. When the antibacterial agent is the injection, the compound K67 in the antibacterial agent has an effective dose of preferably 10 μM to 20 μM, more preferably 20 μM. There is no special limitation on the preparation method of the antibacterial agent, and any preparation method of the antibacterial agent well known in the art can be used.

In an example of the present disclosure, 20 μM of the compound K67 is added into a culture system of the attenuated strain RFP-H37Ra of M. tb. The results show that the number of colonies in the K67 group is significantly reduced compared with the control group, indicating that K67 has a direct antibacterial effect.

In order to further illustrate the present disclosure, the use of a compound K67 in preparation of an anti-tuberculosis drug provided by the present disclosure is described in detail below in connection with accompanying drawings and examples, but these examples should not be understood as limiting the claimed scope of the present disclosure. Sources of materials and reagents

Human M. tb standard attenuated strain H37Ra (CAT: 9302025, purchased from National Center for Medical Culture Collections (CMCC)), M. tb attenuated strain H37Ra expressing red fluorescent protein (RFP-H37Ra) (containing kanamycin resistance gene) referring to the prior art Jie Zhou, Fang Fang, Jinying Qi, et al. Activation of Nrf2 modulates protective immunity against Mycobacterium tuberculosis infection in THP1-derived macrophages. Free Radical Biology and Medicine, 193 (2022) 177-189. Donated by Professor Li Baiqing (Bengbu Medical University, Anhui Key Laboratory of Chronic Disease Immunology and Clinical Medicine);

Human acute monocytic leukemia cells (THP-1, TIB-202) purchased from ATCC (Manassas, USA);

K67: 2-acetyl-1,4-di[(4-methoxyphenylsulfonyl)amino]naphthalene, product number: SML1922, powdered substance, soluble in DMSO, purchased from Sigma-Aldrich;

PMA: phorbol 12-myristate-13-acetate, product number: P1585, powdered substance, soluble in DMSO, purchased from Sigma-Aldrich.

### EXAMPLE 1

### K67 Safety Evaluation

### Methods for Macrophage Culture and Differentiation

THP-1 cells were revived and added into RPMI 1640 medium containing 10% fetal bovine serum (FBS) and a mixture of penicillin/streptomycin/gentamicin antibiotics (final concentrations of penicillin: 100 U/mL, streptomycin: 0.1 mg/mL, gentamicin: 50 μg/mL), and then cultured in a constant-temperature incubator at 37° C. with 5% CO₂. THP-1 cells in desirable cell condition were selected, and when the cell density reached 5×10⁶ cells/mL, they were divided into cell culture plates according to the groups. 100 ng/mL PMA was added into each well to stimulate THP-1 cells for 24 h, such that the THP-1 cells differentiated into adherent spindle-shaped macrophages (THP-1-derived macrophages). After washing with PBS to remove the residual medium, PMA, and undifferentiated THP-1 cells, the cells were transferred to RPMI 1640 medium containing 10% FBS and adjusted to a cell density of 1×10⁶ cells/mL for later use.

### Detection of the Effect of Compound K67 on the Activity of THP-1-Derived Macrophages

The cell activity was detected by CCK8 assay: different doses of K67 were added into the THP-1-derived macrophage test wells (containing 10 μL of THP-1-derived macrophages prepared in Example 1, 10⁴ cells/well) to make the final concentrations of 10 μM, 20 μM, 40 μM, and 80 μM (the solvent was DMSO), and an equal volume of the DMSO was added to a control group, incubated at 37° C. for 24 h, and 10 μL of CCK-8 solution was added (avoiding the generation of air bubbles), and the absorbance at 450 nm was measured with a microplate reader after incubation in a 37° C. incubator for 3 h, and a cell viability was calculated according to the following Formula I.

$$Cell viability = [ ( A s - Ab ) / ( Ac - A b ) ] \times 100 \% \qquad Formula I$$

In Formula I, As represented the absorbance of the experimental well (containing cells, medium, CCK-8 solution, and drug solution), Ac represented the absorbance of the control well (containing cells, medium, and CCK-8 solution but not containing drugs), and Ab represented the absorbance of the blank well (containing medium and CCK-8 solution but not containing cells or drugs).

### Statistical analysis was conducted using GraphPad 9.1.0.

CCK8 assay results showed that there was no statistically significant difference in cell activity between the K67 group and the control group at concentrations of 10 μM and 20 μM (P>0.05, FIG. 1), indicating that within a certain drug concentration range (0-20 μM), the K67 was non-toxic to macrophages and had desirable safety for use.

### EXAMPLE 2

### Extracellular K67 Antibacterial Assay

An inoculation loop of the attenuated M. tb strain RFP-H37Ra was inoculated into Sutong medium (including 2 g/L citric acid, 0.05 g/L ammonium ferric citrate, 1.025 g/L magnesium sulfate, 0.5 g/L potassium dihydrogen phosphate, 4 g/L asparagine, 0.06% (v/v) glycerol, and 50 μg/mL kanamycin) and cultured in a 37° C. constant-temperature bacterial incubator for 3 to 4 weeks. When the RFP-H37Ra in Sutong medium grew to a logarithmic growth phase, 3 to 4 inoculation loops of the strain was inoculated into 300 μL of phosphate-buffered saline-Tween buffer (PBST solution) for thorough grinding, centrifuged at 1,500 rpm for 5 min, the supernatant was pipetted into a 1.5 mL EP tube, and those operations were repeated 3 times to obtain a single bacterial suspension. The live bacteria concentration of the bacterial suspension was measured by the absorbance at a wavelength of 600 nm using a spectrophotometer, and the concentration was adjusted to 1×10⁹ CFU/mL for later use.

A K67 solution (solvent: DMSO) with a final concentration of 20 μM was added into 1 mL of bacterial suspension and cultured in a 37° C. bacterial incubator for 12 h. The bacterial suspension was diluted 100 times with PBST, and 100 μL of the dilution was spread on an agar plate containing kanamycin (50 μg/mL), placed upright for 30 min, and then inverted for culture, which was counted as a K67 group. The difference from the K67 group was that in the control group, the K67 solution was replaced with an equal volume of DMSO solution. After 3 weeks, the growth of M. tb colonies on agar plate was observed and photographed, and colony counting and statistical analysis were conducted.

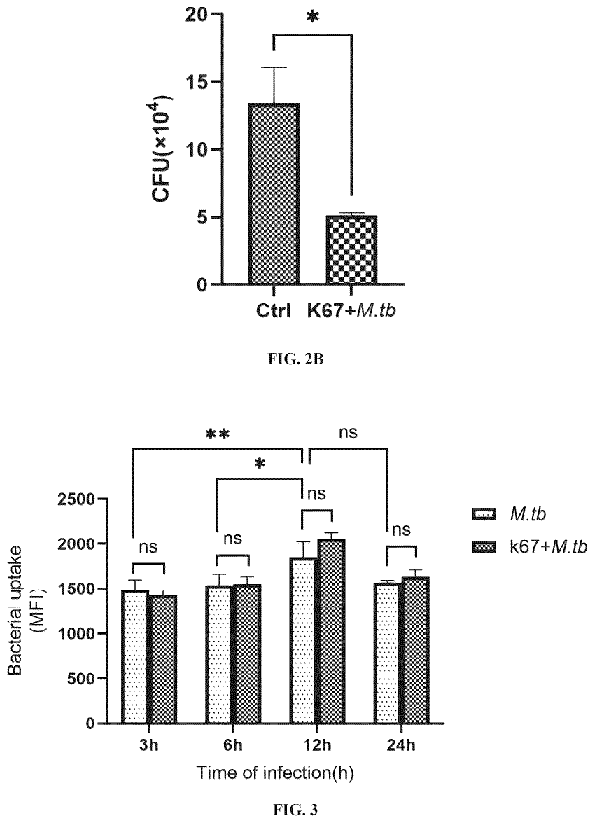

The results showed that compared with the control group, the number of colonies in the K67 group was significantly reduced, and the difference was significant (P<0.05, FIG. 2), indicating that K67 had a direct antibacterial effect.

### Example 3

Effect of K67 on the phagocytic function of macrophages

Method for infection of THP-1-derived macrophages by M. tb

Preparation of H37Ra bacterial suspension and RFP-H37Ra bacterial suspension: an inoculation loop of the attenuated M. tb strain H37Ra was inoculated into Sutong medium (including 2 g/L citric acid, 0.05 g/L ammonium ferric citrate, 1.025 g/L magnesium sulfate, 0.5 g/L potassium dihydrogen phosphate, 4 g/L asparagine, 0.06% (v/v) glycerol) and cultured in a 37° C. constant-temperature bacterial incubator for 3 to 4 weeks. For M. tb expressing red fluorescent protein (RFP-H37Ra), 50 μg/mL of kanamycin was additionally added into the medium. When the M. tb grew to a logarithmic growth phase, 3 to 4 inoculation loops of the strain was inoculated into 300 μL of phosphate-buffered saline-Tween buffer (PBST solution) for thorough grinding, centrifuged at 1,500 rpm for 5 min, the supernatant was pipetted into a 1.5 mL EP tube, and those operations were repeated 3 times to obtain a single bacterial suspension. The live bacteria concentration of the bacterial suspension was measured by the absorbance at a wavelength of 600 nm using a spectrophotometer. The concentration of the bacterial suspension was adjusted to obtain 1×10⁹ CFU/mL of H37Ra bacterial suspension and 1×10⁹ CFU/mL of RFP-H37Ra bacterial suspension for later use.

The THP-1-derived macrophages were infected at MOI=10 (5×10⁶ CFU/5×105 cells) to establish a bacterial infection model.

The treatment method of the K67 group included: 0.5 mL of the THP-1-derived macrophages prepared in Example 1 were added with K67 at a final concentration of 20 μM and cultured for 1 h, and then 5 μL of the RFP-H37Ra bacterial suspension was added to infect the THP-1-derived macrophages at an MOI of 10 (5×10⁶ CFU/5×10⁵ cells), and then cultured for 3 h, 6 h, 12 h, and 24 h, respectively, and 300 μL of trypsin digestion solution containing 0.25% EDTA was added into the cells and incubated in a 37° C. incubator for 5 min. After the incubation, 1 mL of medium without three antibiotics was added into each well to terminate the digestion. The digested cells were transferred to the corresponding flow cytometry tubes, centrifuged at 1,500 rpm for 5 min, a supernatant was discarded, and the cells were resuspended in 300 μL PBS. The above washing operation was repeated 2 times, 300 μL of PBS was added into each tube to resuspend the cells, flow cytometry was conducted to detect the FL3 positive area, and the MFI was recorded and statistically analyzed. The difference from the K67 group was that in the control group, the K67 solution was replaced with an equal volume of DMSO solution.

The results showed that there was no statistically significant difference in MFI between the K67 group and the control group at 3 h, 6 h, 12 h, and 24 h (P>0.05, FIG. 3). This indicated that K67 did not affect the phagocytic function of THP-1-derived macrophages against M. tb.

### EXAMPLE 4

### Effect of K67 Pretreatment on the Killing Activity of Intracellular M. tb in Macrophages

In the K67 group, K67 at a final concentration of 20 μM was added into 0.5 mL of THP-1-derived macrophages prepared in Example 1 and cultured for 1 h, and then 5 μL of the RFP-H37Ra bacterial suspension prepared in Example 3 was added to infect THP-1-derived macrophages at an MOI of 10 (5×10⁶ CFU/5×10⁵ cells), cultured for 12 h, washed 2 times with PBS to remove extracellular K67 and unphagocytosed RFP-H37Ra, and the cells were lysed with 0.05% SDS solution. After the above lysate was diluted 10-fold with ultrapure water, 100 μL of the dilution was aspirated and applied to an agar plate containing kanamycin (50 μg/mL), and then cultured upright for 30 min and then inverted. After 3 weeks, the growth of M. tb colonies on agar plate was observed and photographed, and colony counting and statistical analysis were conducted. The difference from the K67 group was that in the control group, the K67 solution was replaced with an equal volume of DMSO solution.

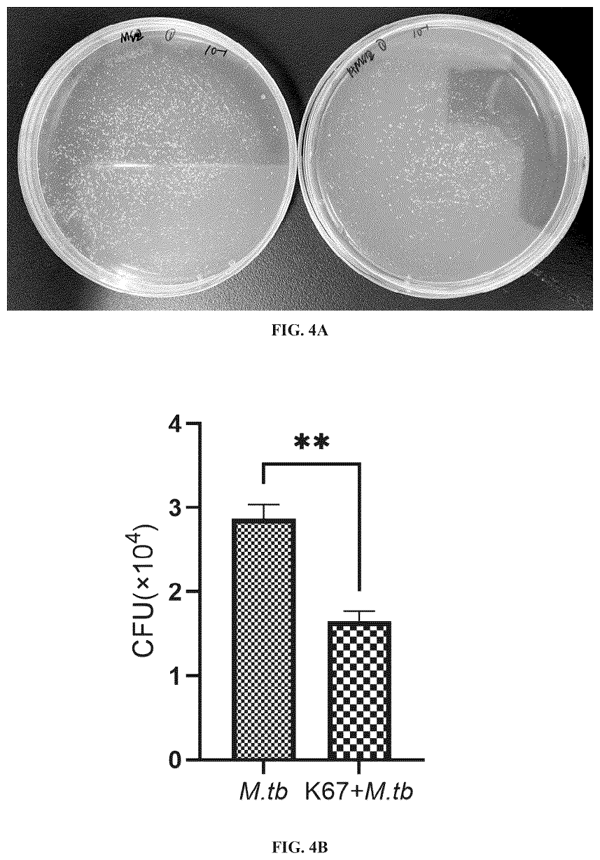

The results showed that compared with the control group, the number of colonies in the K67 group was significantly reduced, with a significant difference (FIG. 4), indicating that K67 could cross the cell membrane into macrophages, act on macrophages that were not infected with M. tb, and regulate the immune response of macrophages to M. tb, thereby effectively killing M. tb that invaded macrophages at the later stage and reducing the load of M. tb in macrophages.

### EXAMPLE 5

### Killing Activity of K67 Against M. tb in Infected Macrophages

5 μL of the RFP-H37Ra bacterial suspension prepared in Example 3 was added, and 0.5mL of THP-1-derived macrophages prepared in Example 1 were infected at MOI=10 (5×10⁶ CFU/5×10⁵ cells), cultured for 12 h, washed 2 times with PBS to remove the unphagocytosed RFP-H37Ra outside the cells, 1640 medium (10% FBS) containing 20 μM K67 was added and cultured for 12 h, washed 2 times with PBS to remove the extracellular K67, and lysed with 0.05% SDS solution. After diluting the lysate 10-fold with ultrapure water, 100 μL of the dilution was aspirated and spread on an agar plate containing kanamycin (50 μg/mL), and cultured upright for 30 min and then inverted. After 3 weeks, the growth of M. tb colonies on agar plate was observed and photographed, and colony counting and statistical analysis were conducted. The difference from the K67 group was that in the control group, the K67 solution was replaced with an equal volume of DMSO solution.

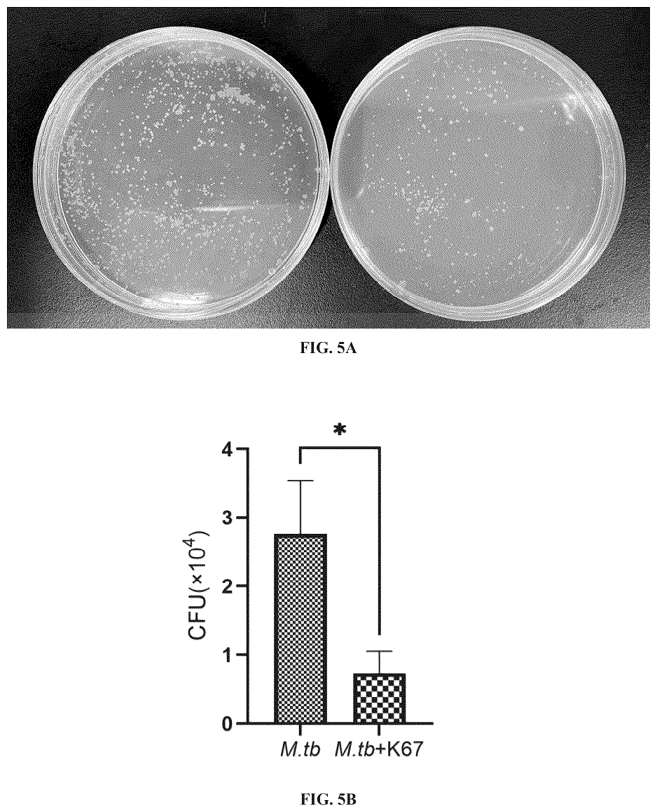

The results showed that compared with the control group, the number of colonies in the K67 group was significantly reduced, with a significant difference (FIG. 5), indicating that K67 could cross the cell membrane and enter into macrophages, act on macrophages that had been infected with M. tb, and change the immune response of infected macrophages to M. tb, thereby effectively killing M. tb that had invaded macrophages and reducing the load of M. tb in macrophages.

### EXAMPLE 6

### Effect of K67 Pretreatment on Autophagy in Macrophages

The experimental groups were set as: a control group, a K67 group, an M. tb group, and a K67+M. tb group.

In the K67+M. tb group, K67 at a final concentration of 20 μM was added into 0.5 mL of THP-1-derived macrophages prepared in Example 1 and cultured for 1 h, and then 5 μL of the H37Ra bacterial suspension prepared in Example 3 was added to infect THP-1-derived macrophages at an MOI of 10 (5×10⁶ CFU/5×10⁵ cells), cultured for 12 h, washed 2 times with PBS to remove extracellular K67 and unphagocytosed H37Ra. Total cell protein was extracted, and Western blot was conducted to analyze the effect of K67 on the expression levels of autophagy-related proteins.

The total cell protein was extracted and the protein concentration was measured by BCA method. After boiling, the protein was placed in a −80° C. refrigerator. SDS-PAGE gel of appropriate concentration was prepared according to the molecular weight of the protein, and the samples were loaded, electrophoresed, wet-transferred, blocked, and incubated with primary antibodies (β-actin, p62, p-p62, and LC3) in a shaker at 4° C. overnight. After washing the membrane three times with TBST, the membrane was incubated with the secondary antibodies (1:5000) corresponding to the species of the primary antibodies at room temperature for 2 h, and then developed with an ECL ultrasensitive luminescent solution and the developed image was collected. Image J software was used to conduct relative quantitative analysis on a gray value of the developed bands.

The sources of primary antibodies were as follows: β-actin Antibody was purchased from Wuhan Sanying, catalog number 20536-1-AP;

SQSTM1/p62 (D1Q5S) Rabbit mAb was purchased from Cell Signaling Technology, catalog number #39749;

Phospho-P62 (Ser349) Antibody was purchased from Wuhan Sanying, catalog number 29503-1-AP;

LC3A/B Antibody was purchased from Cell Signaling Technology, catalog number #12741;

The sources of secondary antibodies were as follows: horseradish peroxidase (HRP)-labeled goat anti-rabbit IgG (H+L) was purchased from Beyotime (Shanghai), catalog number A0208.

Compared with the K67+M. tb group, THP-1-derived macrophages in the K67 group were not infected with H37Ra.

Compared with the K67+M. tb group, the K67 solution in the M. tb group was replaced with an equal volume of DMSO solution.

Compared with the K67+M. tb group, the K67 solution in the control group was replaced with an equal volume of DMSO solution and THP-1-derived macrophages were not infected with H37Ra.

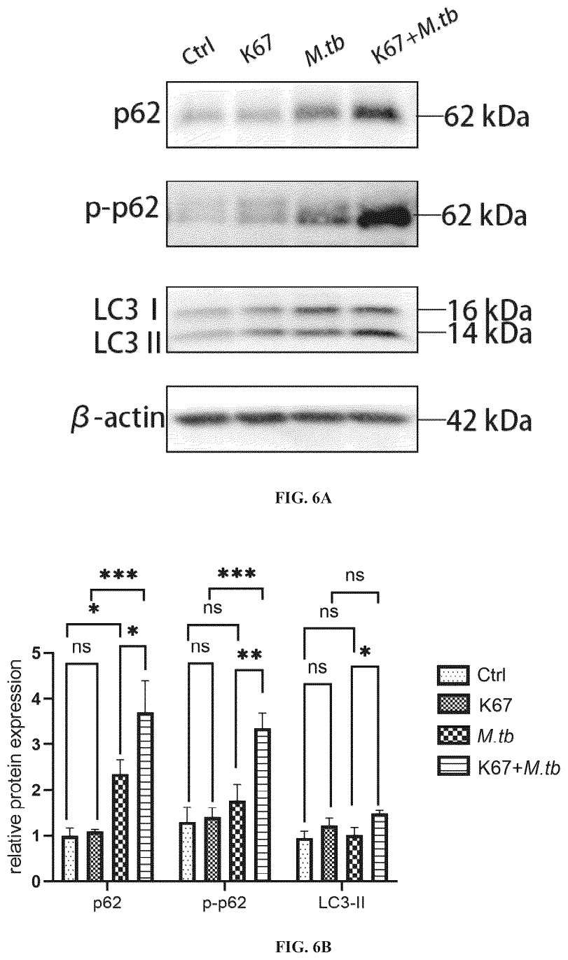

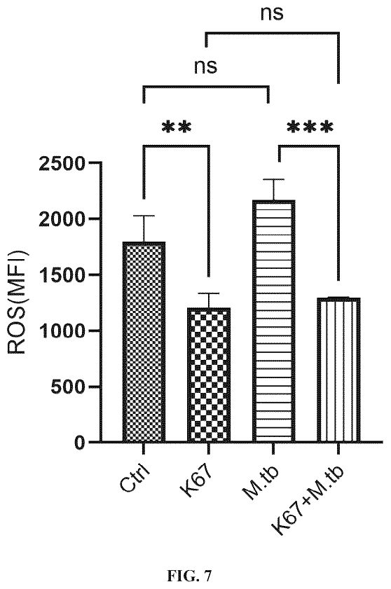

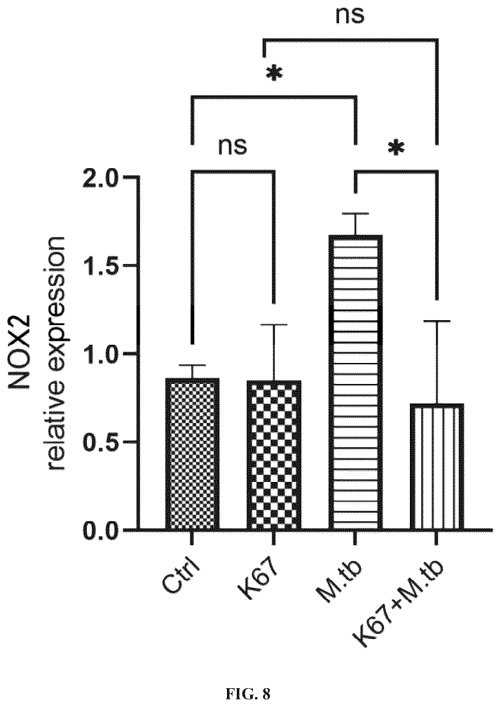

The results showed that compared with the control group, there was no significant difference in the expression of autophagy-related proteins in the K67 group alone (P>0.05); while the expression of autophagy-related proteins in the K67+M. tb group was significantly higher than that in the M. tb infection group alone (FIG. 6). This indicated that the compound K67 could enhance the autophagy level of macrophages infected with M. tb, improve the immune response of cells against M. tb, and facilitate the clearance of intracellular M. tb.

### EXAMPLE 7

### Effect of K67 Pretreatment on ROS Level in Macrophages

The experimental groups were set as: a control group, a K67 group, an M. tb group, and a K67+M. tb group.

The K67+M. tb group was as follows: K67 with a final concentration of 20 μM was added into 0.5 mL of THP-1-derived macrophages prepared in Example 1 and cultured for 1 h, and then 5 μL of the H37Ra bacterial suspension prepared in Example 3 was added to infect THP-1-derived macrophages at an MOI of 10 (5×10⁶ CFU/5×10⁵ cells), cultured for 12 h, and the cells were digested in corresponding flow tubes using a trypsin digestion solution containing 0.25% EDTA, washed 2 times with PBS, centrifuged at 1,500 rpm for 5 min, the supernatant was discarded, and the fluorescent probe DCFH-DA (1:70000) diluted with basic 1640 was added to reach a final concentration of 0.14 μM, 1 mL per tube, placed in a 37° C. water bath for 20 min, and the flow tube was shaken every 3-5 min to allow the cells and the probe to fully contact. After the incubation was completed, the cells were centrifuged at 1,500 rpm for 5 min and wash with ice PBS, the supernatant was discarded for the last time, resuspended with 300 μL ice PBS and placed on an ice box for flow cytometry. The flow cytometry adopted the FL1 channel to record the MFI, and the experimental results were analyzed using FlowJo software. The fluorescent probe DCFH-DA was sourced from the ROS Kit of Beyotime (Shanghai), catalog number: S0033S.

Compared with the K67+M. tb group, THP-1-derived macrophages in the K67 group were not infected with H37Ra.

Compared with the K67+M. tb group, the K67 solution in the M. tb group was replaced with an equal volume of DMSO solution.

Compared with the K67+M. tb group, the K67 solution in the control group was replaced with an equal volume of DMSO solution and THP-1-derived macrophages were not infected with H37Ra.

The results of flow cytometry detection of ROS levels showed that K67 could inhibit the production of ROS in macrophages regardless of whether they were infected with M. tb, with a significant difference (FIG. 7). This indicates that K67 can reduce the oxidative stress level caused by macrophages themselves and M. tb infection to cells, and play a protective role on cells.

### EXAMPLE 8

Effect of K67 pretreatment on the mRNA expression level of NADPH oxidase 2 (NOX2) in macrophages

### 1. Detection of Gene Expression in THP-1-Derived Macrophages

Cells were lysed with Trizol and total RNA was extracted, and the concentration and purity of total RNA were measured using a Thermo Scientific™ NanoDrop™ One micro-volume UV-Vis spectrophotometer. 2 μg of RNA was quantified based on the RNA concentration and the total RNA loading volume was calculated, where a reverse transcription reaction system was as follows:

TABLE 1
Reverse transcription reaction system

| Component | System | |
|---|---|---|
| Total RNA | 0.92 | μL |
| 5 × TransScript ® Uni All-in-One SuperMix for qPCR | 4 | μL |
| gDNA Remover | 1 | μL |
| RNase-free Water | 14.08 | μL |
| Total system | 20 | μL |

After adding the sample, the sample was mixed gently to allow reverse transcription PCR: incubation at 50° C. for 5 min; incubation at 85° C. for 5 s to inactivate EasyScript® RT/RI and gDNA Remover. The product was diluted 5-fold and stored at −80° C. for qPCR.

The primers used in this experiment were designed and synthesized by Shanghai Sangon. The specificity of the primer sequences had been verified by BLAST. The concentration of the primers after dissolution was 100 μM. When being used, the primers were diluted 10-fold to 10 μM with nuclease-free water. The single-stranded DNA template was amplified by qPCR using Roche Light Cycler 480 real-time fluorescence quantitative PCR instrument. Each sample was run in triplicate, and the expression of the target gene was determined by normalization using a reference gene as the standard.

TABLE 2
Primer sequences

| | | Annealing |
|---|---|---|
| Primer name | Primer sequence | temperature |
| GAPDH | For 5′-GAAGGTGAAGGTCGGAGTC-3′ (SEQ ID NO: 1) | 53° C. |
| (Reference | Rev 5′-GAAGATGGTGATGGGATTTC-3′ (SEQ ID NO: 2) | |
| gene) | | |
| NOX2 | For 5′-GCAATGGTGTGAATCGCAGAGTG-3′ (SEQ ID NO: 3) | 56° C. |
| | Rev 5′-TCGAAGACAACTGGACAGGAATCTC-3′ (SEQ ID NO: 4) | |
| TNF-α | For 5′-TGGCGTGGAGCTGAGAGATAAC-3′ (SEQ ID NO: 5) | 57° C. |
| | Rev 5′-GCTGATGGTGTGGGTGAGGAG-3′ (SEQ ID NO: 6) | |
| IL-6 | For 5′-TTCGGTCCAGTTGCCTTCTCC-3′ (SEQ ID NO: 7) | 56° C. |
| | Rev 5′-TCTGAAGAGGTGAGTGGCTGTC-3′ (SEQ ID NO: 8) | |
| IL-1β | For 5′-ATGGCTTATTACAGTGGCAATGAGG-3′ (SEQ ID NO: 9) | 55° C. |
| | Rev 5′-AGTGGTGGTCGGAGATTCGTAG-3′ (SEQ ID NO: 10) | |

TABLE 3
qPCR system

| Component | System | |
|---|---|---|
| Template | 2 | μL |
| Forward primer (10 μM) | 0.4 | μL |
| Reverse primer (10 μM) | 0.4 | μL |
| 2 × PerfecStart ®Green qPCR SuperMix | 10 | μL |
| Nuclease-free Water | 7.2 | μL |
| Total system | 20 | μL |

TABLE 4
qPCR instrument program setting

| Program | Temperature and time | Number of cycles |
|---|---|---|
| Initial denaturation | 94° C., 30 s | 1 |
| Three-step program | 94° C., 5 s | 45 |
| | 50-60° C., 15 s | |
| | 72° C., 10 s | |
| Melting | — | 1 |
| Cooling | — | 1 |

The annealing temperature in the three-step method was set according to the Tₘ of the primers; in addition, the default settings of the instrument, without modification, were used for the melting and cooling programs.

### Analysis Method

The CT values of the reference genes and target genes in each group were exported using the LightCycler480 analysis software, and the mean CT value of each group was calculated. The mean CT value of the reference gene was used to normalize the mean CT value of the target gene, that is, ΔCT=CT (mean value of target gene)−CT (mean value of reference gene). The ΔCT value of the target gene in the control group was calculated in three repeated experiments. The ΔCT value of the control group was used to normalize the ΔCT value of each group, that is, ΔΔCT=ΔCT (control and treatment groups)−ΔCT (control group), and finally the 2^{-ΔΔCT} value was calculated.

The experimental groups were set as follows: a control group, a K67 group, an M. tb group, and a K67+M. tb group.

In the K67+M. tb group, K67 at a final concentration of 20 μM was added into 0.5 mL of THP-1-derived macrophages prepared in Example 1 and cultured for 1 h, and then 5 L of the H37Ra bacterial suspension prepared in Example 3 was added to infect THP-1-derived macrophages at an MOI of 10 (5×10⁶ CFU/5×10⁵ cells), cultured for 12 h, washed 2 times with PBS to remove extracellular K67 and unphagocytosed H37Ra, total cell RNA was extracted and RT-qPCR was conducted to detect the expression level of NOX2 mRNA.

Compared with the K67+M. tb group, THP-1-derived macrophages in the K67 group were not infected with H37Ra.

Compared with the K67+M. tb group, the K67 solution in the M. tb group was replaced with an equal volume of DMSO solution.

Compared with the K67+M. tb group, the K67 solution in the control group was replaced with an equal volume of DMSO solution and THP-1-derived macrophages were not infected with H37Ra.

The results showed that compared with the control group, there was no significant difference in the expression of NOX2 mRNA in the group treated with K67 alone (P>0.05); M. tb infection led to up-regulation of NOX2 mRNA expression, which was significantly different (P<0.05); when K67 was present, K67 would down-regulate the high expression of NOX2 mRNA caused by M. tb infection, which was significantly different (P<0.05, FIG. 8). This indicated that the compound K67 could reduce the expression level of NOX2 in macrophages and inhibit the oxidative stress response caused by M. tb infection.

### EXAMPLE 9

Effect of K67 pretreatment on the expression levels of pro-inflammatory cytokines TNF-α, IL-6, and IL-1β in macrophages

The experimental groups were set as follows: a control group, a K67 group, an M. tb group, and a K67+M. tb group.

In the K67+M. tb group, K67 at a final concentration of 20 μM was added into 0.5 mL of THP-1-derived macrophages prepared in Example 1 and cultured for 1 h, and then 5 μL of the H37Ra bacterial suspension prepared in Example 3 was added to infect THP-1-derived macrophages at an MOI of 10 (5×10⁶ CFU/5×10⁵ cells), cultured for 12 h. The cell culture supernatant was aspirated, and the expression levels of the pro-inflammatory cytokines were detected by ELISA. The cells were washed 2 times with PBS to remove extracellular K67 and unphagocytosed H37Ra, total cell RNA was extracted by the method in Example 8, and the expression levels of mRNA of the pro-inflammatory cytokines were detected by RT-qPCR.

The ELISA kit for human TNF-α was purchased from R&D Systems, catalog number DY210-05;

The ELISA kit for human interleukin-6 (IL-6) was purchased from R&D Systems, catalog number DY206;

The ELISA kit for human interleukin-1β (IL-1β) was purchased from Jiangsu Jingmei Biotechnology Co., Ltd., catalog number JM-03336H1.

The operation method of ELISA included: coating, blocking, and washing were conducted; samples and standards, detection antibodies, colorimetric substrates, and stop solution were added, and the absorbance was detected at 450 nm with an ELISA reader and a standard curve was plotted to calculate the sample concentration.

Compared with the K67+M. tb group, THP-1-derived macrophages in the K67 group were not infected with H37Ra.

Compared with the K67+M. tb group, the K67 solution in the M. tb group was replaced with an equal volume of DMSO solution.

Compared with the K67+M. tb group, the K67 solution in the control group was replaced with an equal volume of DMSO solution and THP-1-derived macrophages were not infected with H37Ra.

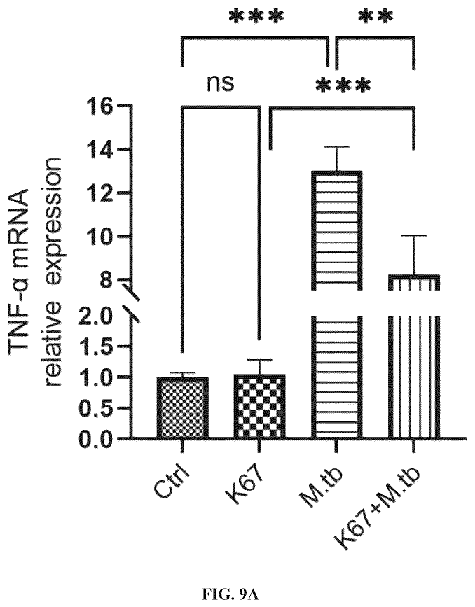

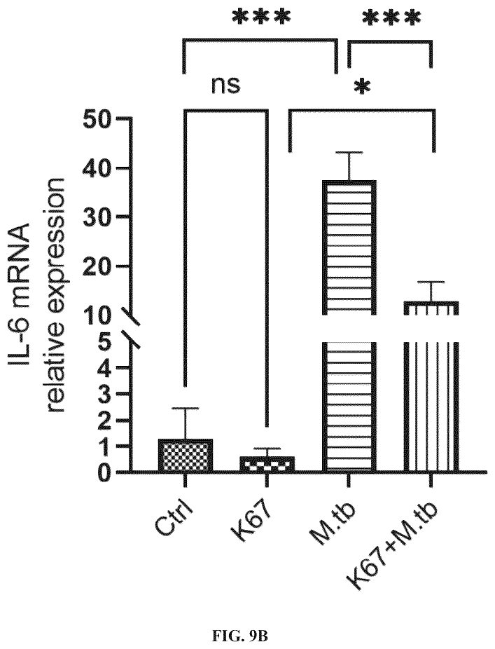

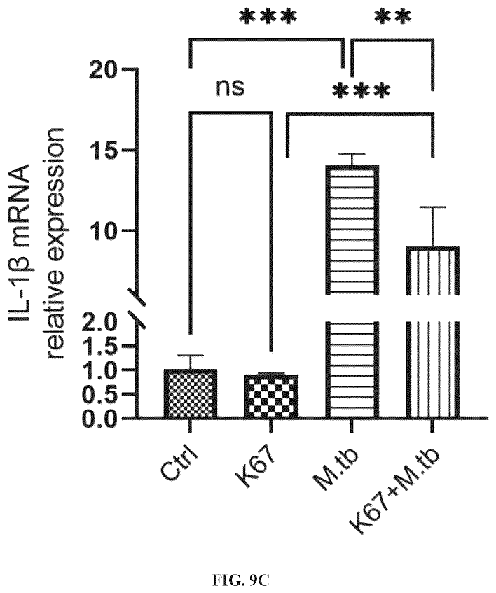

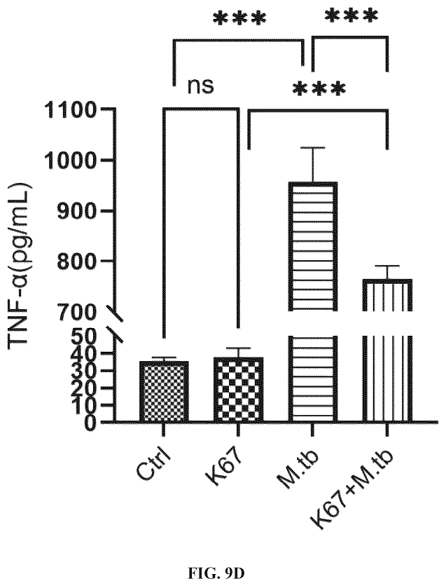

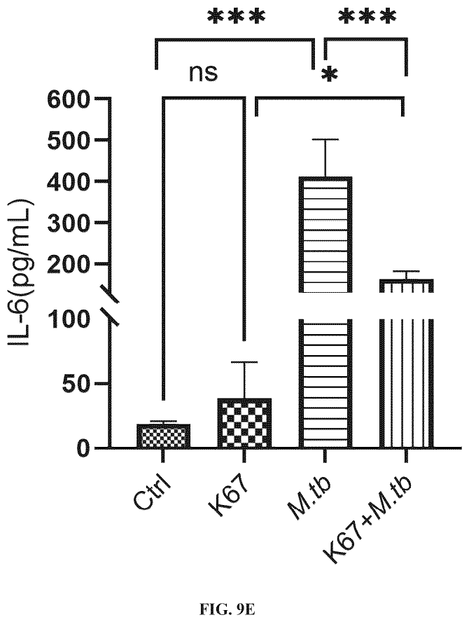

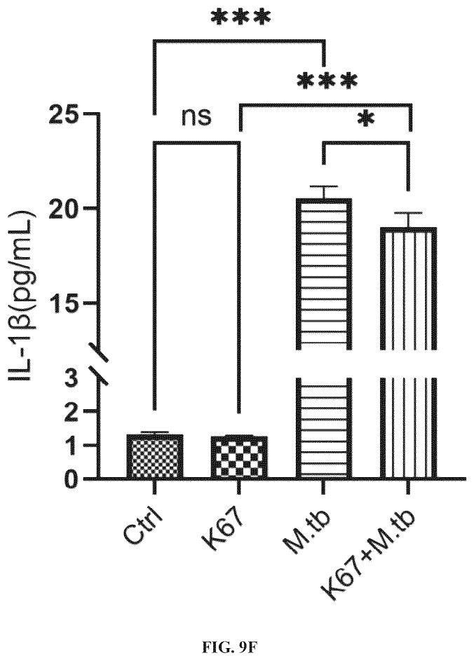

The results showed that compared with the control group, there was no significant difference in the mRNA and protein expression levels of TNF-α, IL-6, and IL-1βin the K67 treatment group alone (P>0.05); while M. tb infection led to an increase in the mRNA and protein expression levels of TNF-α, IL-6, and IL-1β, with significant differences (P<0.001). When K67 was present, K67 could down-regulate the high expression of mRNA and protein of various pro-inflammatory cytokines caused by M. tb infection, with significant differences (FIG. 9). This suggests that the compound K67 can inhibit the high inflammatory state caused by M. tb infection and protect cells from damage by oxidative stress.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A method for treating tuberculosis, comprising administering a medicament comprising a compound K67 to a patient in need thereof;
wherein the compound K67 is 2-acetyl-1,4-di[(4-methoxyphenylsulfonyl)amino]naphthalene having a structure shown in Formula I:

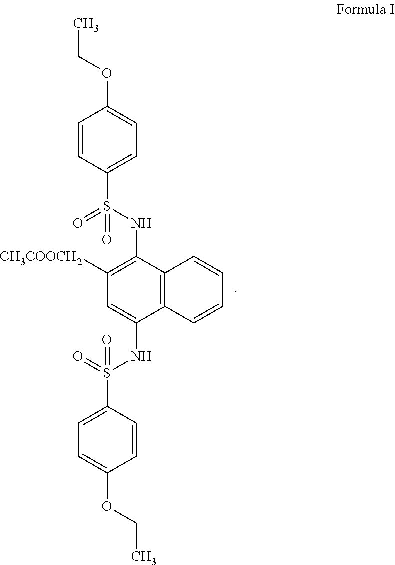

2. The method according to claim 1, further comprising inhibiting proliferation of Mycobacterium tuberculosis in a body.

3. The method according to claim 1, wherein the compound K67 regulates at least one of the following reactions: increasing an autophagy level of macrophages, reducing an oxidative stress level of the macrophages, and inhibiting an inflammatory response in the macrophages; and
the macrophages are Mycobacterium tuberculosis-infected macrophages.

4. The method according to claim 3, wherein the increasing the autophagy level of the macrophages comprises promoting expression of an autophagy-related protein and/or increasing an LC3 II/LC3 I ratio.

5. The method according to claim 3, wherein the reducing the oxidative stress level of the macrophages comprises reducing a level of reactive oxygen species (ROS) in the macrophages and/or reducing an expression level of nicotinamide adenine dinucleotide phosphate (NADPH) oxidase 2 (NOX2) in the macrophages.

6. The method according to claim 3, wherein the inhibiting the inflammatory response in the macrophages comprises inhibiting expression of a gene and/or a protein of a pro-inflammatory factor in the macrophages.

7. The method according to claim 6, wherein the pro-inflammatory factor is at least one selected from the group consisting of tumor necrosis factor (TNF)-α, interleukin (IL)-6, and IL-1β.

8. An antibacterial agent for inhibiting a mycobacterium, comprising a compound K67, wherein the compound K67 is 2-acetyl-1,4-di[(4-methoxyphenylsulfonyl)amino]naphthalene having a structure shown in Formula I:

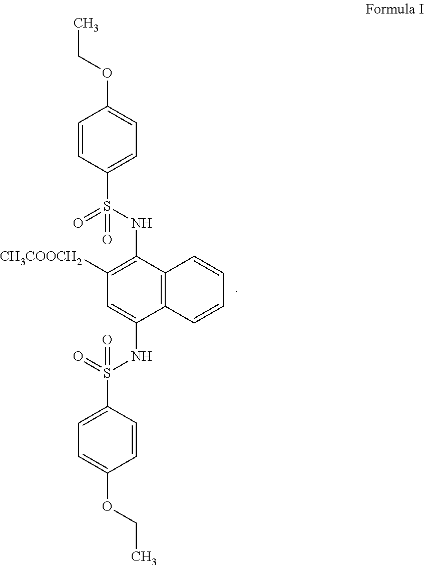

9. The antibacterial agent according to claim 8, wherein the mycobacterium is Mycobacterium tuberculosis.
